(19) United States
(12) Patent Application Publication
(10) Pub. No.: US 2026/0230741 A1
(43) Pub. Date: Aug. 6, 2026

(54) MANAGING AIRFLOW WITH A CONTROLLABLE AIRWAY EARBUD SYSTEM

(71) Applicant: MOTOROLA MOBILITY LLC, WILMINGTON, DE (US)

(72) Inventors: AMIT KUMAR AGRAWAL, BANGALORE (IN); AMAL CHANDRAN, BENGALURU (IN)

(21) Appl. No.: 19/042,603

(22) Filed: Jan. 31, 2025

(51) Int. Cl. H04R 1/10; A61B 5/00; A61B 5/01; G06F 3/16; G10L 13/02; G10L 25/78

(52) U.S. Cl. CPC H04R 1/1041; A61B 5/01; A61B 5/6817; G06F 3/165; H04R 1/1016; G10L 13/02; G10L 25/78; H04R 2460/11

(57) ABSTRACT

A method provides techniques for monitoring, on an electronic device that includes a communications subsystem enabling the electronic device to communicatively connect to a controllable airway earbud (CAE) device, one or more in-ear usage parameters corresponding to usage of the CAE device by a user. In response to a monitored in-ear usage parameter exceeding a respective parameter threshold, an airflow control message is transmitted to the CAE device to trigger an adjustment in a seal of the CAE device and enable airflow into a corresponding ear canal of the user. The CAE device is configured with a fabric seal providing a seal against the ear canal that can be dynamically modified.

### BACKGROUND

### 1. Technical Field

The present disclosure generally relates to electronic devices utilized for audio playback, and more specifically to wireless earbuds.

### 2. Description of the Related Art

Using wireless earbuds with a smartphone or tablet computer offers several advantages for both listening to audio and engaging in voice conversations. Wireless earbuds eliminate the need for cords/cables, allowing a user to move freely without having to be concerned about the cord/cable getting tangled or breaking when exercising, commuting, or performing general tasks. With no wires connected between the earbuds and an associated audio source, such as a smartphone, wireless earbuds are generally more comfortable to wear for extended periods. Moreover, wireless earbuds can provide advanced audio technologies, such as active noise cancellation (ANC), spatial audio, or enhanced bass for an immersive listening experience. Furthermore, for voice conversations, wireless earbuds can enable speaking on the telephone/cellphone while keeping hands free for other activities, such as typing, cleaning, or cooking. By combining convenience, portability, and advanced audio features, wireless earbuds greatly enhance the experience of using smartphones and tablets for both entertainment and communication.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1A presents a functional block diagram of example components of an electronic device in a communication environment and having hardware and software components that enable the features of the present disclosure to be advantageously implemented, according to one or more embodiments;

FIG. 1B is an additional block diagram representation of the electronic device of FIG. 1A presenting additional components, including components for wireless communications with other devices, according to one or more embodiments;

FIG. 2A illustrates exemplary components of a controllable airway earbud system used in a sealed configuration during media playback that reduces airflow into the inner ear canal, according to one or more embodiments;

FIG. 2B illustrates an example controllable airway earbud system in an unsealed configuration while media is paused, increasing airflow into the inner ear, according to one or more embodiments;

FIG. 2C illustrates an example controllable airway earbud system with an electronically controllable smart fabric seal in a standard/normal configuration, according to one or more embodiments;

FIG. 2D illustrates an example controllable airway earbud system with an electronically controllable smart fabric seal in a retracted configuration to enable increased ventilation, according to one or more embodiments;

FIG. 3A is a diagram illustrating the sealed state of a controllable airway earbud system based on varying audio levels, according to one or more embodiments;

FIG. 3B is a diagram illustrating the sealed state of a controllable airway earbud system based on audio levels and an inserted media pause event to extend the duration of an unsealed state, according to one or more embodiments;

FIG. 4A shows an exemplary smart fabric that can be used to create a seal for a controllable airway earbud system, in an undeformed configuration, according to one or more embodiments;

FIG. 4B shows the exemplary smart fabric of FIG. 4A in a deformed configuration to enable airflow, based on application of a trigger input, according to one or more embodiments;

FIG. 5 illustrates an exemplary user interface for controllable airway earbud system configuration, according to one or more embodiments;

FIG. 6 depicts a flowchart of a computer-implemented method for managing airflow with a controllable airway earbud system, according to one or more embodiments;

FIG. 7 depicts a flowchart of a computer-implemented method for managing airflow with a controllable airway earbud system based on ear canal temperature, according to one or more embodiments;

FIG. 8 depicts a flowchart of a computer-implemented method for managing airflow with a controllable airway earbud system based on in-ear duration, according to one or more embodiments;

FIG. 9 depicts a flowchart of a computer-implemented method for managing airflow with a controllable airway earbud system based on detected pauses in media playback, according to one or more embodiments;

FIG. 10 depicts a flowchart of a computer-implemented method for managing airflow with a controllable airway earbud system based on audio state data, according to one or more embodiments; and

FIG. 11 depicts a flowchart of a computer-implemented method for managing airflow with a controllable airway earbud system based on silence breaks between audio tracks, according to one or more embodiments.

### DETAILED DESCRIPTION

According to aspects of the present disclosure, an electronic device, a method, and a computer program product provide techniques for adjusting in-ear airflow via a controllable airway earbud (CAE) device. A CAE device is one that provides a mechanism for controlling how much and/or whether air is able to pass to and from the inner ear canal. The CAE device can include vents, and/or adjustable seals, and/or other mechanisms for enabling a change in an amount of airflow that can occur between the outer ear and the inner ear. In one or more embodiments, the adjustable seals can be comprised of a smart fabric that deforms when an electrical signal is applied thereto, to enable a change to the fit of the seals against the walls of the inner ear to allow or restrict airflow.

Wireless earbuds typically come in pairs, with one earbud for each ear. Each earbud can include a speaker, which is the transducer that converts electrical signals into audible sound waves. Additionally, the wireless earbuds may each include one or more microphones, which can be used for voice commands, receiving audible speech during phone calls, and/or noise cancellation. The earbuds may each further include a Bluetooth transceiver to handle the wireless communication with a paired electronic device such as a smartphone. The Bluetooth transceiver can include both a transmitter and a receiver to send and receive signals. The earbuds further include a processor for managing tasks such as sound processing, Bluetooth communication, and sensor data processing. The earbuds may each include a battery, such as a lithium-ion battery, to provide power for operating all the components within the earbuds. The earbuds may further include a seal to provide a snug and comfortable fit in the ear of a user. The seals can also serve to provide some noise isolation. For example, the seals help block outside noises, and can create an improved listening experience. Moreover, the noise reduction provided by the seals can augment active noise cancellation (ANC) techniques that are implemented in wireless earbuds. In addition, the wireless earbuds may include one or more sensors, including, but not limited to, optical sensors, accelerometers, gyroscopes, infrared sensors, pressure sensors, and/or temperature sensors. Sensors such as infrared sensors and/or optical sensors can be configured to detect when the earbuds are being worn in the ears of a user.

While wearing wireless earbuds provide a useful convenience to device users, wearing earbuds for extended periods can indeed pose several risks to ear health. In particular, earbuds can trap moisture and bacteria in the ear canal, creating an environment conducive to infections, which can cause pain, as well as temporary or even permanent loss of hearing.

The disclosed embodiments address the aforementioned issues caused by prolonged use of wireless earbuds by communicating with a CAE device to increase the airflow between the ambient environment (region surrounding the person) and the inner ear by adjusting a seal of the CAE device. According to one aspect, the disclosed embodiment takes into consideration the fact that increased airflow can also allow ambient noise to be more easily detected by the person, which can create an undesirable user experience. Disclosed embodiments utilize audio information, such as an audio state of a user as speaking or non-speaking, as well as the state of media being played back, to determine an optimal time to adjust the seal and trigger the increased airflow. In one or more embodiments, when playing audio tracks, such as songs, the increase in airflow is scheduled to occur during a detected gap of silence in between songs. When audio resumes, e.g., with playing of the next track/song, the seal is opened to decrease, minimize, or eliminate the airflow in order to improve noise isolation from the ambient environment. In another embodiment, when the CAE device is being used for audio calls, when the audio state of the user (wearer of the CAE device) is speaking, the seal is adjusted, such that the airflow into the inner ear canal is increased. When the audio state of the user is non-speaking, which typically correlates to the user listening to incoming audio, the seal is again extended such that airflow to the inner ear canal from the ambient environment is decreased, to improve sound quality. In this way, disclosed embodiments enable increased airflow to the inner ear canal when conditions are such that the increased airflow is less noticeable to a user. Similarly, disclosed embodiments decrease the airflow to the inner ear canal when conditions are such that increased airflow would be more noticeable and could create a negative user experience. By intelligently controlling the amount of airflow allowed to pass the CAE device, disclosed embodiments can improve ear health and safety when using CAE devices while still providing an optimal listening experience in terms of noise blocking and/or noise cancellation during audio reception by the CAE device.

According to one aspect, one or more in-ear usage parameters corresponding to use of a CAE device by a user are monitored. The in-ear usage parameters can include in-ear duration (how long the CAE devices have been in the ears of a user), inner ear temperature, inner ear pressure, inner ear humidity, and/or other in-ear usage parameters. In response to one or more of the in-ear usage parameters exceeding a respective parameter threshold, an airflow control message is sent to the CAE device to trigger an adjustment in a seal of the CAE device and enable airflow into a corresponding ear canal of the user.

One or more embodiments can provide an electronic device that includes: a communications subsystem enabling the electronic device to communicatively connect to a controllable airway earbud (CAE) device; a media subsystem enabling the electronic device to send transmitted audio data to and to receive audio data from the CAE device; a memory having stored thereon an ear health monitoring (EHM) module; and at least one processor coupled to the communications subsystem, the media subsystem, and the memory and which processes program code of the EHM module. The at least one processor is configured to cause the electronic device to: monitor one or more in-ear usage parameters corresponding to usage of the CAE device by a user; and in response to a monitored in-ear usage parameter exceeding a respective parameter threshold, transmit an airflow control message to the CAE device to trigger an adjustment in a seal of the CAE device and enable airflow into a corresponding ear canal of the user.

One or more embodiments can provide a method that includes: monitoring, on an electronic device that includes a communications subsystem enabling the electronic device to communicatively connect to a controllable airway earbud (CAE) device, one or more in-ear usage parameters corresponding to usage of the CAE device by a user; and in response to a monitored in-ear usage parameter exceeding a respective parameter threshold, transmitting an airflow control message to the CAE device to trigger an adjustment in a seal of the CAE device and enable airflow into a corresponding ear canal of the user.

Further embodiments can provide a computer program product including: a non-transitory computer readable medium; and program code on the computer readable medium that when processed by a processor of an electronic device configures the processor to perform functions of the above-described method.

The above descriptions contain simplifications, generalizations and omissions of detail and is not intended as a comprehensive description of the claimed subject matter but, rather, is intended to provide a brief overview of some of the functionality associated therewith. Other systems, methods, functionality, features, and advantages of the claimed subject matter will be or will become apparent to one with skill in the art upon examination of the figures and the remaining detailed written description. The above as well as additional objectives, features, and advantages of the present disclosure will become apparent in the following detailed description.

Each of the above and below described features and functions of the various different aspects, which are presented as operations performed by the processor(s) of the communication/electronic devices are also described as features and functions provided by a plurality of corresponding methods and computer program products, within the various different embodiments presented herein. In the embodiments presented as computer program products, the computer program product includes a non-transitory computer readable storage device having program instructions or code stored thereon, and configuring the electronic device and/or host electronic device to complete the functionality of a respective one of the above-described processes when the program instructions or code are processed by at least one processor of the corresponding electronic/communication device, such as is described above.

In the following description, specific example embodiments in which the disclosure may be practiced are described in sufficient detail to enable those skilled in the art to practice the disclosed embodiments. For example, specific details such as specific method orders, structures, elements, and connections have been presented herein. However, it is to be understood that the specific details presented need not be utilized to practice embodiments of the present disclosure. It is also to be understood that other embodiments may be utilized and that logical, architectural, programmatic, mechanical, electrical and other changes may be made without departing from the general scope of the disclosure. The following detailed description is, therefore, not to be taken in a limiting sense, and the scope of the present disclosure is defined by the appended claims and equivalents thereof.

References within the specification to “one embodiment,” “an embodiment,” “embodiments”, “some embodiments”, or “one or more embodiments” are intended to indicate that a particular feature, structure, or characteristic described in connection with the embodiment is included in at least one implementation (embodiment) of the present disclosure. The appearance of such phrases in various places within the specification are not necessarily all referring to the same embodiment, nor are separate or alternative embodiments mutually exclusive of other embodiments. Further, various features are described which may be exhibited by some embodiments and not by others. Similarly, various aspects are described which may be aspects for some embodiments but not for other embodiments.

The terminology used herein is for the purpose of describing particular embodiments only and is not intended to be limiting of the disclosure. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Moreover, the use of the terms first, second, etc. do not denote any order or importance, but rather the terms first, second, etc. are used to distinguish one element (e.g., a person or a device) from another.

It is understood that the use of specific component, device and/or parameter names and/or corresponding acronyms thereof, such as those of the executing utility, logic, and/or firmware described herein, are for example only and not meant to imply any limitations on the described embodiments. The embodiments may thus be described with different nomenclature and/or terminology utilized to describe the components, devices, parameters, methods and/or functions herein, without limitation. References to any specific protocol or proprietary name in describing one or more elements, features or concepts of the embodiments are provided solely as examples of one implementation, and such references do not limit the extension of the claimed embodiments to embodiments in which different element, feature, protocol, or concept names are utilized. Thus, each term utilized herein is to be provided its broadest interpretation given the context in which that term is utilized.

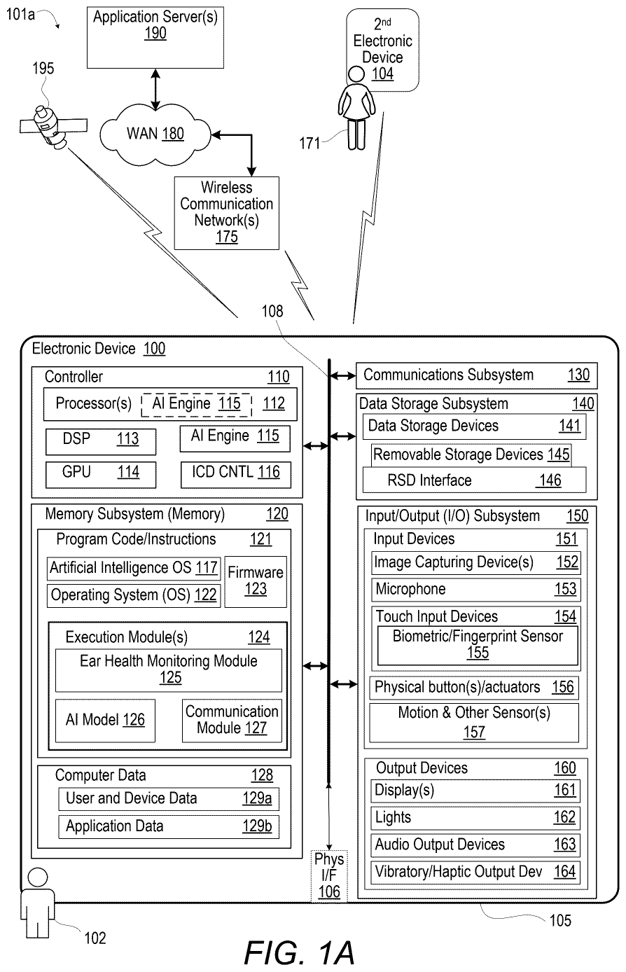

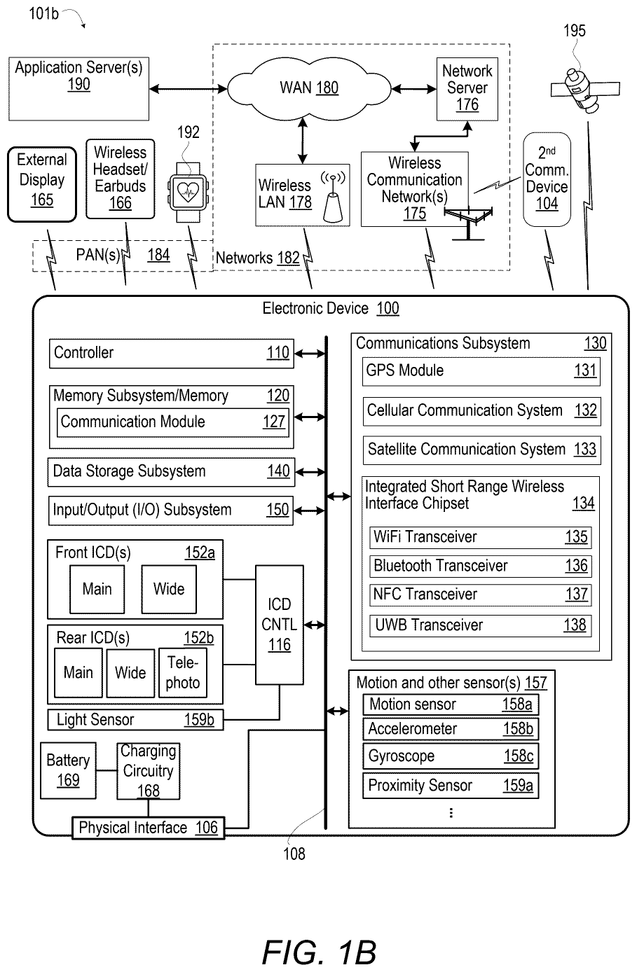

Those of ordinary skill in the art will appreciate that the hardware components and basic configuration depicted in the following figures may vary. For example, the illustrative components within electronic device 100 (FIG. 1A-1B) are not intended to be exhaustive, but rather are representative to highlight components that can be utilized to implement the present disclosure. For example, other devices/components may be used in addition to, or in place of, the hardware depicted. The depicted example is not meant to imply architectural or other limitations with respect to the presently described embodiments and/or the general disclosure. Throughout this disclosure, the terms ‘electronic device’, ‘communication device’, and ‘electronic communication device’ may be used interchangeably, and may refer to devices such as smartphones, tablet computers, and/or other computing/communication devices.

Within the descriptions of the different views of the figures, the use of the same reference numerals and/or symbols in different drawings indicates similar or identical items, and similar elements can be provided similar names and reference numerals throughout the figure(s). The specific identifiers/names and reference numerals assigned to the elements are provided solely to aid in the description and are not meant to imply any limitations (structural or functional or otherwise) on the described embodiments.

Referring now to the figures and beginning with FIG. 1A, there is illustrated a block diagram of an example electronic device 100 in communication environment 101a and having hardware and software components, which enable the features of the present disclosure to be advantageously implemented, according to one or more embodiments.

Examples of electronic device 100 can include, but are not limited to, mobile devices, a notebook computer, a mobile phone, a smart phone, a digital camera with enhanced processing capabilities, a smart watch, a tablet computer, and other types of electronic devices. For purposes of this disclosure, electronic device 100 is assumed to be a communication device that can be used to engage in a voice and/or video call with a second communication device. Electronic device 100 can therefore be interchangeably referred to herein as communication device 100.

Electronic device 100 generally includes controller 110, memory (or memory subsystem) 120, communication subsystem 130, data storage subsystem 140, input/output subsystem 150, all contained within or extended from an exterior surface of device housing 105. Controller 110 is shown communicatively connected/coupled via system interlink 108 with each of the subsystems 120, 130, 140, and 150, and is directly or indirectly connected with the individual components within each subsystem 120, 130, 140, and 150. System interlink 108 represents internal components that facilitate internal communication by way of one or more shared or dedicated internal communication links, such as internal serial or parallel buses. As utilized herein, the term “communicatively coupled” means that information signals are transmissible through various interconnections, including wired and/or wireless links, between the components. The interconnections between the components can be direct interconnections that include conductive transmission media or may be indirect interconnections that include one or more intermediate electrical components.

Controller 110 includes processor 112, which includes one or more central processing units (CPUs) or data processors. Processor 112 performs many of the features of controller 110 and references to features performed by controller 110 can be interchangeably referred to herein as features of processor 112, and vice-versa. In some embodiments, the various functions associated with controller 110 are integrated into processor 112, and accordingly, references made herein to controller and/or processor are understood to refer to one or both components as providing a single management component within the electronic device 100. For simplicity in describing the features of the electronic device 100, the operational functions provided by one or more of operational components within controller 110, including those provided by processor 112 are collectively described as being performed by controller 110. Collectively, components integrated within controller 110 support computing, classifying, processing, transmitting and receiving of data and information, and presenting of graphical and photographic images within a display.

As illustrated, controller 110 can also include one or more digital signal processors 113 graphics processing units (GPUs) 114, artificial intelligence (AI) engine 115, and image capturing device (ICD) controller 116. In some embodiments, the functionality of each of these additional processing components can be integrated with processor(s) 112. For example, processor 112 can, in some embodiments, include dedicated AI engine 115 and image signal processors (ISPs) (not shown). Processor 112 can further include other processors such as auxiliary processor(s) that may act as a low power consumption, always-on sensor hub for physical sensors.

Controller 110 manages, and in some instances directly controls, the various functions and/or operations of electronic device 100. These functions and/or operations include, but are not limited to including, application data processing, communication, location and navigation tasks, image processing, and signal processing. In one or more alternate embodiments, electronic device 100 may use hardware component equivalents for application data processing and signal processing. For example, electronic device 100 may use special purpose hardware, dedicated processors, general purpose computers, microprocessor-based computers, micro-controllers, optical computers, analog computers, dedicated processors and/or dedicated hard-wired logic. Controller 110 can, in some embodiments, also include a hardware acceleration (HA) unit, which can establish direct memory access (DMA) sessions to route network traffic to various elements within electronic device 100 without direct involvement from processor 112 and/or a device operating system 122. Operating system 122 may include or be augmented by device AI operating system (OS) 117 that can include native support for AI-specific hardware such as Neural Processing Units (NPUs) or Tensor Processing Units (TPUs) to optimize performance for AI tasks such as machine learning inference and training.

Memory subsystem (or memory) 120 may include a combination of volatile and non-volatile memory, such as random-access memory (RAM) and read-only memory (ROM). Memory subsystem 120 stores instruction or program code 121 for execution by processor 112 to configure processor 112 (and more generally electronic device 100) to provide the operational functions and features described herein. Instructions/program code 121 (or program code 121 for short) includes instructions for an operating system (OS) 122, firmware 123, such as basic input/output system (BIOS) or Uniform Extensible Firmware Interface (UEFI). Program code 121 includes execution module(s) 124 that collectively provides the various features of the disclosure. Execution module(s) 124 include, without limitation, ear health monitoring (EHM) module 125, which provides the features and operating functionality of the disclosed embodiments when the corresponding program instructions of ear health monitoring (EHM) module 125 are processed by/within processor 112/controller 110.

Execution modules 124 further includes AI model(s) 126. In one or more embodiments, processor 112 can utilize AI models 126 to provide AI functionality of processor-integrated AI engine 115. In other embodiments, AI models 126 are directly utilized by AI engine 115. In one or more embodiments, AI model(s) 126 is integrated as a sub-module within EHM module 125 and is trained to support AI features of EHM module 125. AI model(s) 126 may include an artificial neural network, a decision tree, a support vector machine, Hidden Markov model, linear regression, logistic regression, Bayesian networks, and so forth. AI model(s) 126 can be individually trained to perform specific tasks and can be arranged in different sets of AI models to generate different types of output. Training of AI model(s) 126 is the process by which AI models are trained to perform specific tasks or achieve certain objectives. The training involves providing the model with a large amount of data and allowing the model to learn from patterns and relationships within that data.

Each of the above-introduced module(s) and/or application(s) provides program instructions/code that are processed by processor 112 and which configures processor 112 (and/or controller 110) and/or other operational components of electronic device 100 to cause the electronic device 100 to perform specific operations and functions, as described herein. Descriptive names assigned to these modules add no functionality and are provided solely to assist in identifying the underlying features performed by processing the different modules. For example, EHM module 125 can include program instructions that cause or configure processor 112 to cause electronic device 100 to adjust the airflow of a controllable airway earbud system based on input audio data and/or ambient audio data. Other features provided by EHM module 125 are described in further detail throughout this disclosure.

Program code 121 can further include instructions/code for other applications (not shown) providing different features of/within electronic device 100. In one or more embodiments, program code 121 may be integrated into a distinct chipset or hardware module as firmware that operates separately from other executable program code. Portions of program code 121 may be incorporated into different hardware components that operate in a distributed or collaborative manner.

Memory subsystem 120 also includes computer data 128. During execution of program code 121, processor 112 may access, use, generate, modify, store, or communicate computer data 128, such as user and device data 129a and application data 129b. Computer data 128 may incorporate “data” that originated as raw, real-world “analog” information that consists of basic facts and figures. Computer data 128 includes different forms of data, such as numerical data, images, coding, notes, and financial data, as well as data presenting video, graphics, text, and images. Computer data 128 may originate at electronic device 100 or may be retrieved from a remote device via communications subsystem 130. Electronic device 100 may store, modify, present, or transmit computer data 128.

Communications subsystem 130 includes various components that enable electronic device 100 to communicate with external communication networks and other devices, such as second electronic device 104 and application server(s) 190, etc., via communications subsystem 130. According to one or more embodiments, communication module 127 presented within program code 121 includes instructions supporting the use of communications subsystem 130 to establish communication interfaces enabling communication by electronic device 100 with these external networks and devices.

Data storage subsystem 140 of electronic device 100 includes data storage device(s) 141. Controller 110 is communicatively connected, via system interlink 108, to data storage device(s) 141. Data storage subsystem 140 provides stored versions of program code 121 and computer data 128 on nonvolatile storage that is accessible by controller 110. The program code 121 can be loaded into memory 120 for execution/processing by controller 110. In one or more embodiments, data storage device(s) 141 can include hard disk drives (HDDs), optical disk drives, and/or solid-state drives (SSDs), etc.

Data storage subsystem 140 of electronic device 100 can include removable storage device(s) (RSD(s)) 145, which is received in RSD interface 146. Controller 110 is communicatively connected to RSD 145, via system interlink 108 through RSD interface 146. In one or more embodiments, RSD 145 is a non-transitory computer program product or computer readable storage device that stores program code and associated data, including a copy of EHM module 125 and AI model(s) 126, which may be executed by a processor associated with a user device, such as electronic device 100. Controller 110 can access data storage device(s) 141 or RSD(s) 145 to provision electronic device 100 with stored program code 121 and computer data 128 that, when executed/processed by processor 112, the program code configures processor 112 and/or more generally electronic device 100, to provide the various functions described herein.

I/O subsystem 150 includes input devices 151 such as, but not limited to, image capturing device(s) (ICDs) 152, microphone 153, and touch input devices 154 (e.g., touch screens, keys, or buttons) for use by a user to interface with electronic device 100. Touch input devices 154 can include a biometric/fingerprint sensor 155 for biometric input. Biometric/fingerprint sensor 155 can be used to read/receive biometric data, such as fingerprints, to identify or authenticate a user. In some embodiments, the biometric sensor 155 can supplement an ICD (camera), which captures images for user detection/identification via facial recognition.

Input devices 151 may include physical buttons/actuators 156 that can be located on a periphery of the device housing 105. Physical buttons/actuators 156 may provide controls for volume, power, and ICDs 152. Microphone 153 can also be referred to as an audio input device. In some embodiments, microphone 153 may be used for identifying a user via voiceprint, voice recognition, and/or other suitable techniques. Input devices 151 can also include one or more motion or other sensor(s) 157, which are further defined in the FIG. 1B description which follows.

With reference to FIG. 1B, as illustrated, motion and other sensor(s) 157 of electronic device 100 include, but are not limited to, one or more motion sensor(s) 158a, one or more accelerometers 158b, one or more gyroscopes 158c, and proximity sensor 159a, etc. Motion sensor(s) 158a detect movement of electronic device 100 and provide motion data to processor 112 indicating the spatial orientation, position and movement of electronic device 100. Accelerometers 158b measure linear acceleration of movement of electronic device 100 in multiple axes (X, Y and Z). For example, accelerometers 158b can include three accelerometers, where one accelerometer measures linear acceleration in the X axis, one accelerometer measures linear acceleration in the Y axis, and one accelerometer measures linear acceleration in the Z axis. Accelerometers 158b can be used to calculate the orientation/position of electronic device 100 relative to the earth and can also be referred to as a gravity sensor. Gyroscope 158c measures rotation or angular rotational velocity of electronic device 100. Proximity sensor 159a senses the presence of nearby objects. In one embodiment, proximity sensor 159a can be an infrared (IR) sensor that detects the presence of a nearby object, such as when electronic device 100 is in a pocket of a user. Electronic device 100 can also include one or more light sensors 159b, which detects the luminance and/or intensity (i.e., the amount) of ambient light surrounding the electronic device 100.

Referring again to FIG. 1A, I/O subsystem 150 includes output devices 160 such as, but not limited to, display(s) 161, lights 162, audio output devices 163, and vibratory and/or haptic output devices 164. In one or more embodiments, electronic device 100 includes an integrated display 161 which incorporates a tactile, touch screen interface that can receive a user's tactile/touch input. As a touch screen device, integrated display 161 allows a user to provide input to and/or to control electronic device 100 by touching features within a user interface presented on integrated display 161. Tactile, touch input device 154 can include a touch screen interface. The touch screen interface can include one or more virtual buttons or selectable affordances. In one or more embodiments, when a user 102 applies a finger or stylus on the touch screen interface (154) in the region demarked by the virtual button, the touch of the region causes the processor 112 to execute code to implement a function associated with the virtual button. In some implementations, integrated display 161 is integrated into a front surface of electronic device housing 105 along with front image capturing devices (not specifically shown), while the higher quality ICDs are located on a rear surface of device housing 105. Other embodiments provide multiple integrated displays within electronic device 100 and references to display(s) 161 are assumed to refer to one or all of these multiple integrated displays.

Vibration/haptic output device 164 can cause electronic device 100 to vibrate or shake when activated. Vibration/haptic output device 164 can be activated during an incoming call or message in order to provide an alert or notification to a user of electronic device 100. In one or more embodiments, integrated display 161, audio output devices (or speakers) 163, and vibration/haptic device 164 can generally and collectively be referred to as output devices.

With reference again to FIG. 1B and with continuing reference to FIG. 1A, there is presented another view of electronic device 100 with components enabling electronic device 100 to function as a mobile communication device, within an expanded communication environment 101b. In addition to the functional and operational components already presented by and described within the description of FIG. 1A, FIG. 1B further illustrates expanded communications subsystem 130 with additional communication components and interfaces enabling electronic device 100 to perform wireless communications within an expanded communication environment 101b that includes other devices.

Communications subsystem 130 includes global positioning system (GPS) module 131 that enables electronic device 100 to communicate with and receive GPS location data from GPS satellite(s) 195. In one or more embodiments, GPS module 131 receives geospatial input from GPS broadcasts of time data and location data from GPS satellite(s) 195 to obtain geospatial location information about the physical location of electronic device 100.

In one or more embodiments, controller 110, via communications subsystem 130, performs multiple types of cellular over-the-air (OTA) or non-cellular wireless communication, such as by using a Bluetooth connection or other personal access network (PAN) connection. As shown, communications subsystem 130 includes cellular communication system 132, which includes at least one radio frequency RF front end coupled to one or more antennas. In one or more embodiments, cellular communication system 132 can include a communication module with one or more baseband processors or digital signal processors, one or more modems, and a radio frequency (RF) front end having one or more transmitters and one or more receivers. In one or more embodiments, controller 110, via communications subsystem 130, may communicate via an OTA cellular connection with radio access networks (RANs) over a cellular wireless communication network (CWCN) 175. CWCN 175 can be a terrestrial network and include a plurality of base stations and associated network server(s) 176, in one embodiment. Cellular communication system 132 allows electronic device 100 to communicate wirelessly with CWCN 175 via transmissions of communication signals (represented as lightning bolts) to and from network communication devices, such as base stations or cellular nodes, of CWCN 175. Alternatively, or in addition, CWCN 175 can include a satellite network, and electronic device 100 connects to CWCN 175 using satellite communication system 133. Cellular communication system 132 and satellite communication system 133 enable electronic device 100 to engage in long distance wireless communication capabilities.

In one or more embodiments, communications subsystem 130 includes integrated short range wireless interface chipset 134 having one or more of Wi-Fi transceiver (TxRX) 135, Bluetooth (BT) TxRx 136, near field communication (NFC) transceiver 137, and ultra-wideband (UWB) transceiver 138. In one or more embodiments, the short-range communication devices are not integrated on a single chipset but can be separately provided hardware components. In one or more embodiments, electronic device 100 can communicate wirelessly with external wireless devices, such as a Wi-Fi router of a wireless local area network (WLAN) 178 and/or second electronic device 104, via one or more short-range wireless interface(s). Second electronic device 104 can be a communication device, such as a smartphone, and/or can be similarly configured as electronic device 100. Second user 171 may operate second electronic device 104. In one or more embodiments, electronic device 100 can receive Internet or Wi-Fi based calls, text messages, multimedia messages, and other notifications via a combination of wireless and wired networks (generally networks 182).

In one or more embodiments, networks 182 can include CWCN 175, WLAN 178, and Wide Area Network (WAN) 180, such as the Internet. In one or more embodiments, WAN 180 can enable electronic device 100 to access application servers 190, which can provide a downloadable version of EHM module 125 and/or access to other applications, online transactions, and resources. In one or more embodiments, networks 182 can also include personal area networks (PAN) 184, which are individually created with second devices via one of short-range wireless devices from among Wi-Fi TxRX 135, BT TxRx 136, NFC transceiver 137, and UWB transceiver 138. Example second devices include external display 165, wireless headset (or earbuds) 166, and wearable computing device 192. External display 165 can be a stand-alone monitor/display or a display integrated into a second electronic device, such as a laptop computer. In at least one embodiment, connection to the external display 165 can be wired and can include an intermediate connection device, such as a docking station device. In one or more embodiments, wearable computing device 192, such as a smartwatch, fitness tracker, or the like, may be paired with electronic device 100, and provide biometric data such as heart rate, breathing rate, and the like, to the electronic device 100 via the paired communication link.

Electronic device 100 also includes a physical interface 106. Physical interface 106 of electronic device 100 can serve as an input/output data port and can be used as a power supply port that is coupled to charging circuitry 168 which feeds electrical power to device battery 169 to enable recharging of device battery 169 and/or powering of electronic device 100. As a data port, physical interface 106 can enable electronic device 100 to be physically coupled via a cable or docking station port to a second device, such as external display 165.

FIG. 1B also presents additional details of ICD(s) 152 of electronic device 100. Throughout the disclosure, the term image capturing device (ICD) is synonymous with and/or utilized interchangeably with any one of the cameras of electronic device 100. ICD(s) (or cameras) 152 includes front cameras 152a and rear cameras 152b. In one embodiment, each of front cameras 152a and rear cameras 152b are communicatively coupled to ICD controller 116. ICD controller 116 supports the processing of image data from front cameras 152a and rear cameras 152b. Front cameras 152a can include a main camera and a wide-angle camera. Rear cameras 152b can include a main camera, a wide-angle camera, and a telephoto camera. Both sets of cameras 152 include image sensors that can capture images that are within the field of view (FOV) of each respective camera 152. In one or more embodiments, one or more of the cameras can be utilized to enable biometric authentication using facial image and/or iris scan recognition.

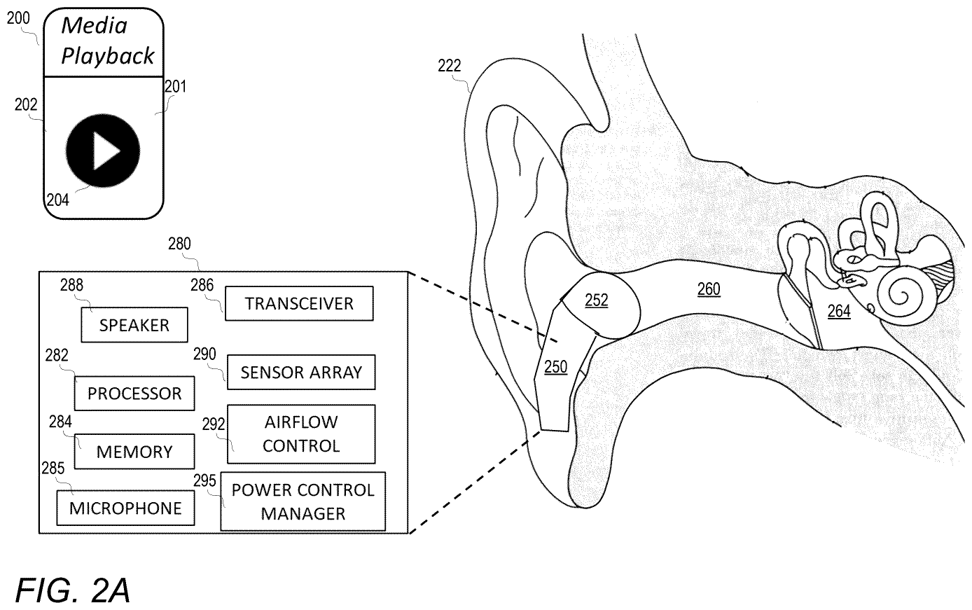

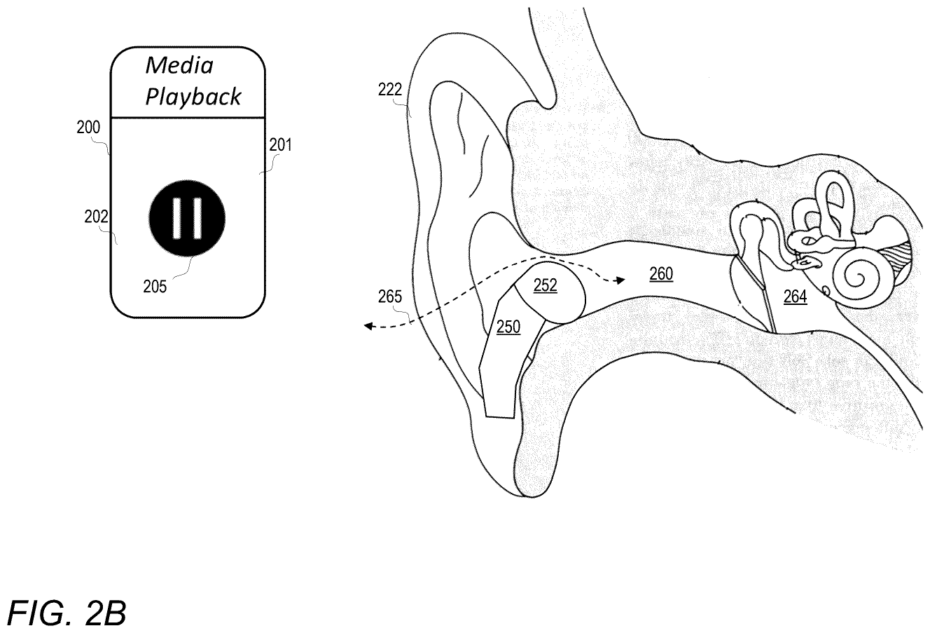

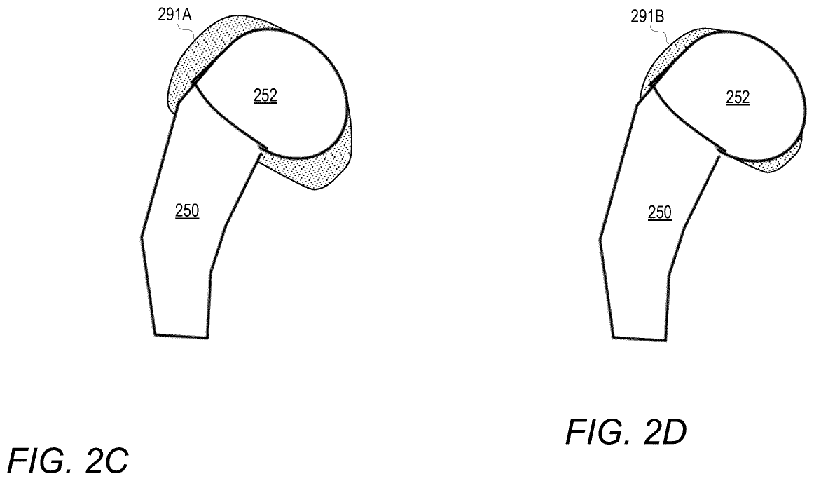

FIG. 2A illustrates an example controllable airway earbud system operating in a sealed configuration during media playback, according to one or more embodiments. An example electronic device 200 is shown having display 202. Rendered and presented on display 202 is a user interface 201 for a media playback application (app) that indicates playing of media at 204. Device 200 may be similar to electronic device 100 depicted in FIG. 1. The electronic device 200 is communicatively coupled to CAE device 250. In one or more embodiments, the electronic device 200 is communicatively coupled to CAE device 250 via Bluetooth pairing. The communicative coupling between electronic device 200 and CAE device 250 enables audio data to be wirelessly sent from electronic device 200 to CAE device 250 and vice versa. In one or more embodiments, the audio data is transmitted using the A2DP (Advanced Audio Distribution Profile) and/or Auracast protocol. The audio data may be compressed utilizing the SBC (Subband coding) codec prior to transmission.

As can be seen in FIG. 2A, the CAE device 250 fits within the ear and includes an exterior seal 252 that forms a boundary between the inner ear canal 260 and the auricle 222. Accordingly, the ambient environment (air outside the auricle) is substantially sealed from the inner ear 264 by seal 252. Seal 252 may be comprised of silicone, rubber, and/or other suitable materials, to provide comfort and noise isolation for the user. In one or more embodiments, seal 252 may be comprised of a smart fabric that can change texture when a voltage or current (or other deformation triggering input that can be locally controlled or modulated) is applied to it. Other embodiments may use electronically controllable vents instead of, or in addition to, a smart fabric

A block diagram of main components of CAE device 250 is shown at 280. The CAE device can include a processor 282, which is coupled to memory 284, which contains instructions that are executed by processor 282 to perform one or more functions of the CAE device. The CAE device 250 includes a speaker 288 to produce soundwaves based on audio data that is received from the electronic device 200 via transceiver 286. The CAE device 250 can further include a microphone 285 to support voice calls, voice commands, noise cancellation, and/or other features. The CAE device 250 can further include a sensor array 290. The sensor array can include one or more sensors, including but not limited to, an infrared sensor, optical sensor, temperature sensor, humidity sensor, and/or other suitable sensors.

The CAE device further includes an airflow control component 292. The airflow control component 292 can include one or more controllable open/close vents. The CAE device can further include a power control manager 295 that includes components to provide the electronic signaling and/or logic for controlling the vents. Alternatively, and/or in addition, the airflow control component can comprise a smart fabric within the seal 252 (e.g., the seal can be made of the smart fabric) that can deform when an electrical signal is applied, in order to change the fit of the seal 252 against the inner ear canal 260 to enable control of airflow to the inner ear 264. In one or more embodiments, the power control manager 295 includes the electronic signaling and/or logic for controlling a smart fabric. One or more embodiments can include: detecting a first characteristic of the CAE device that enables reconfiguration of at least one external surface (i.e., reconfiguration of the external seal) of the CAE device to allow air flow past the CAE device into the corresponding ear canal; and selecting an airflow control message to send to the CAE device to adjust the seal of the CAE device, in response to detecting the first characteristic. According to one or more embodiments, the seal is adjusted by contracting the fabric to make the external surface of the seal smaller relative to the ear canal. According to one or more embodiments, the seal is adjusted by causing the fabric to move away from one surface of the ear canal.

FIG. 2B illustrates an example controllable airway earbud system in an unsealed configuration while media is paused, according to one or more embodiments. As shown in FIG. 2B, the user interface 201 indicates at 205 that the media has been paused. In response to the pausing of the media (e.g., by the device user), the electronic device 200 sends an airflow control message to the CAE device to trigger an adjustment in a seal of the CAE device and enable airflow into a corresponding ear canal of the user. The airflow control message can include a discrete value. In one or more embodiments, an integer value of zero in the airflow control message can cause the CAE device to reduce airflow, while an integer value of one in the airflow control message can cause the CAE device to increase airflow. In one or more embodiments, values received in an airflow control message are used to trigger the power control manager 295 to generate the appropriate electronic signals to control the ventilation accordingly. One or more embodiments may utilize other discrete values to enable finer grained control of the amount of ventilation/airflow provided by the CAE device. In response to receiving the airflow control message, the processor of the CAE device (282 of FIG. 2A) causes the seal 252 to be deformed to enable airflow 265 into the inner ear canal 260. The airflow 265 serves to reduce moisture in the inner ear canal 260 that can accumulate while wearing tight fitting earbuds.

Avoiding excess moisture in the ears is crucial for preventing infections because moisture creates an environment conducive to the growth of bacteria and fungi, which can lead to infections like otitis externa (commonly known as swimmer's ear). Bacteria thrive in warm, moist environments. Excess moisture in the ear canal can break down the skin's protective barrier, allowing bacteria to penetrate and cause infections. Thus, disclosed embodiments serve to reduce moisture in the inner ear canal, which can reduce the risk of infections, and promote overall ear health.

FIG. 2C shows an example of a CAE device 250 having a smart fabric 291A disposed on an exterior of the earbud seal 252. In one or more embodiments, the smart fabric is disposed around an outer surface of the earbud seal 252. In one or more embodiments, the smart fabric 291A can be configured to change texture when a voltage is applied thereto. The change in texture from smooth to varied can enable air to pass from the ambient environment to the inner ear canal, reducing the temperature of the inner ear canal and/or reducing humidity levels, to promote ear health.

FIG. 2D shows the CAE device 250 of FIG. 2C with the smart fabric 291B configured to change shape/texture as compared to as shown in FIG. 2C, to enable increased ventilation as shown in FIG. 2B. The increased ventilation also increases the amount of outside noise that is noticeable by the user. Accordingly, disclosed embodiments strategically increase the ventilation under conditions in which the increased ventilation is less likely to have noticeable adverse effects in terms of sound quality. Smart fabric 291A can be the same as smart fabric 291B, with the necessary voltages applied to change the texture and/or volume of the seal.

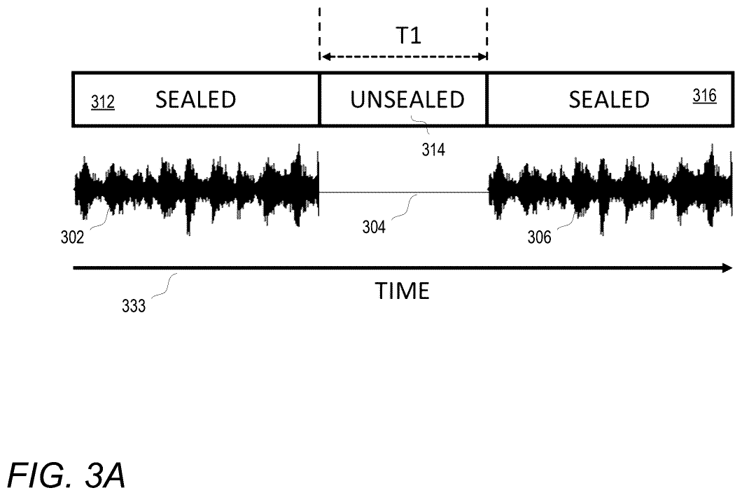

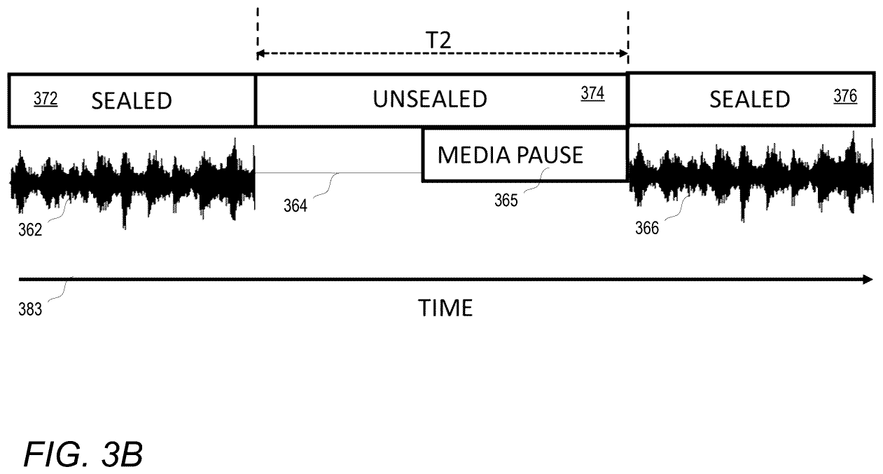

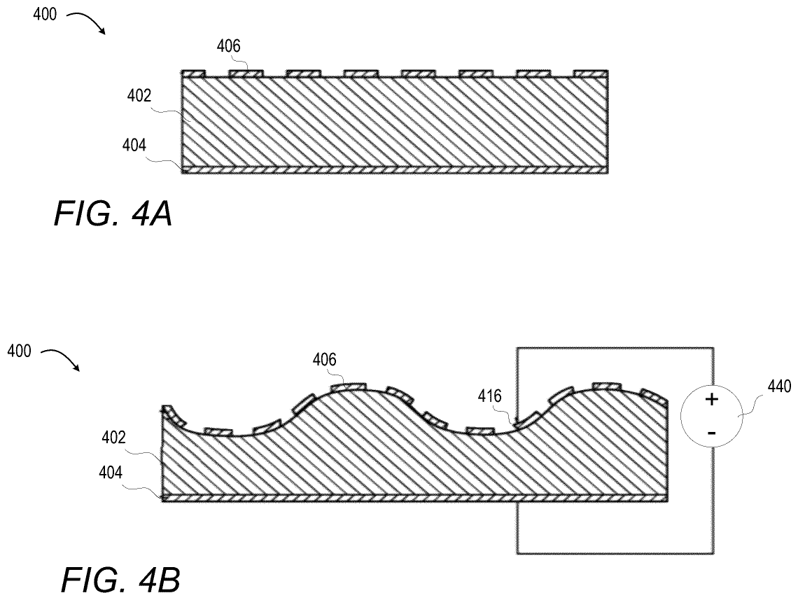

FIG. 3A is a diagram illustrating the seal state of a controllable airway earbud system based on current audio levels, according to one or more embodiments. A time axis is depicted with a horizontal arrow labeled 333, pointing to the right, indicating the direction of increasing time. A first segment of audio is shown at 302, and a corresponding seal state of the CAE of sealed is shown at 312. A seal state of sealed is shown in FIG. 2A with seal 252 (comprising external fabric or material in expanded form) sealing the inner ear canal 260. A segment of silence (audio break) is shown at 304, and a corresponding seal state of the CAE of unsealed is shown at 314. A seal state of unsealed is shown in FIG. 2B with seal 252 deformed to allow airflow 265 into the inner ear canal 260. A second segment of audio is shown at 306, and a corresponding changed seal state of CAE of sealed is shown at 316. Accordingly, during the silence period indicated at 304, the CAE device is configured to reduce the substantially full seal of the inner ear canal to enable air to ventilate the inner ear canal, thereby reducing moisture in the inner ear canal, while also not adversely affecting the listening experience, since the ventilation occurs when no sound is being outputted by the CAE device. The period of silence indicated at 304 can occur when one audio track ends, but before a next audio track starts, such as when listening to a playlist of songs. As can be seen, the unsealed state 314 has a duration T1. In one or more embodiments, the duration T1 can range from 500 milliseconds to 1500 milliseconds. Other time duration values are possible, and the presented values are for illustration only.

FIG. 3B is a diagram illustrating the state of a controllable airway earbud system based on audio levels and an inserted media pause event, according to one or more embodiments. Similar to what is shown in FIG. 3A, a time axis is depicted with a horizontal arrow labeled 383, pointing to the right, indicating the direction of increasing time. A first segment of audio is shown at 362, and a corresponding seal state of CAE device of sealed is shown at 372. A segment of silence is shown at 364, and a corresponding seal state of CAE device of unsealed is shown at 374. However, unlike in FIG. 3A, the unsealed state 374 is extended to a duration indicated by T2, where T2 is greater than T1 of FIG. 3A. In one or more embodiments, the duration T2 can range from 1500 milliseconds to 3000 milliseconds. To enable the increased duration of the unsealed state shown at 374, the electronic device (e.g., 200 of FIG. 2A), inserts media pause event 365. The pause event introduces an additional delay before starting the next audio track, indicated at 366. When the next audio track starts, the electronic device (e.g., 200 of FIG. 2A), sends an airflow control message to the CAE device to cause the CAE device to return to the sealed state, as indicated at 376.

As an example, the default silence period between audio tracks can be 1 second, and the media pause event can be 1 second, to effectively create a silence period of 2 seconds. The increased silence period enables the extended unsealed state as indicated at 374, while avoiding an adverse listening experience of the unsealed state while audio playback is occurring. In one or more embodiments, in response to detecting an elevated inner ear temperature, elevated inner ear humidity level, an extended period of wearing the CAE device uninterrupted, and/or other conditions, the processor is configured to cause the electronic device to insert a media pause event to increase the silence period, enabling increased ventilation of the inner ear canal without an undesirable increase in outside noise during audio playback. In this way, disclosed embodiments can serve to increase airflow to the inner ear, thereby reducing infection risk and improving overall ear health, while providing an enjoyable and comfortable user experience when using wireless earbuds that are configured to support/enable controllable airflow.

FIG. 4A shows an exemplary smart fabric 400, which can be used in CAE devices in accordance with one or more embodiments. FIG. 4A shows an undeformed structure. The smart fabric 400 can include a nanovoid polymer (NVP) element 402 which is a flexible material with many, microscopic voids therein, a plurality of electrodes on a first surface of the NVP element, indicated generally as electrode 406, and a common electrode 404 disposed on a second surface of the NVP element. In embodiments, a control circuit may be used to apply variable voltages to one or more of the plurality of electrodes on the upper surface of the NVP element.

FIG. 4B shows the smart fabric 400 of FIG. 4A after creating a spatially varying deformation of the NVP element 402 based on a trigger input that includes applying a voltage to each of the plurality of electrodes. For simplicity, FIG. 4B shows a voltage source 440 that may be used to apply the voltage between one or more electrodes of the plurality of electrodes and the common electrode. The applied voltage causes the electrodes on the first surface to be attracted to the common electrode. This attraction arises because opposite charges create an electric field between the electrodes, resulting in a Coulomb force. The Coulomb force pulls the electrodes toward each other, as oppositely charged objects attract. By spatially varying the applied voltage, a texture can be formed on the smart fabric that causes voids between the CAE device and the surface of the inner ear, enabling increased airflow, as depicted in FIG. 2B. Alternatively, by providing a uniform voltage to one or more electrodes, a volume reduction of the seal can be achieved, thereby increasing ventilation into the inner ear canal as shown in FIG. 2B.

For illustrative clarity, an electrical connection is shown only to a single electrode, indicated as 416. In embodiments, the processor of the CAE device (e.g., 282 of FIG. 2A) may control the voltage to the electrodes via airflow control component 292 (FIG. 2A). The seals of the CAE device can be fabricated using smart fabric 400 of FIG. 4A and FIG. 4B. The airflow can be adjusted by adjusting the voltage applied to the smart fabric. Other embodiments may utilize other ventilation techniques instead of, or in addition to, the smart fabric shown in FIG. 4A and FIG. 4B.

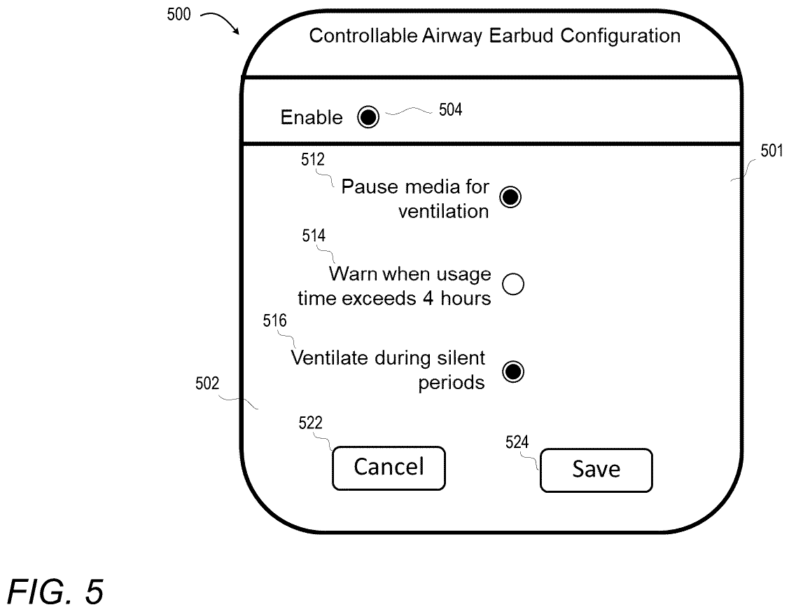

FIG. 5 illustrates an exemplary user interface for controllable airway earbud system configuration, according to one or more embodiments. Device 500 may be similar to electronic device 100 depicted in FIG. 1. Device 500 includes display 502 on which user interface 501 is rendered and presented. The user interface 501 can include an option to enable the controllable airway earbud feature 504, which is indicated as selected in FIG. 5. The user interface 501 can include an option to pause media for ventilation, indicated at 512 as selected. The functionality of pausing media for ventilation, to allow for modifying the state of the seal of the CAE device during the period in which media playback is pause, is depicted in FIG. 3B. The user interface 501 can include an option to issue a warning when a usage time exceeds a predetermined duration (e.g., four hours), indicated at 514, as unselected. In one or more embodiments, if option 514 is selected, and the duration of wearing the CAE device exceeds the predetermined threshold, a warning message is issued. In one or more embodiments, the warning message can include sending an auditory warning message to the CAE device that causes the CAE device to issue an audible alert to the user. The auditory warning message can include a tone or other sound. In one or more embodiments, the auditory warning message can include synthesized speech to alert the user to the excessive duration, and/or encourage the user to take a break from the CAE device, to allow ventilation into the inner ear canal, for the purposes of promoting ear health. The warning message can also be presented as a text message on or overlaying user interface 501. Accordingly, one or more embodiments can include: in response to detecting an ear canal temperature exceeding a second predetermined threshold, sending an auditory warning message to the CAE device that causes the CAE device to issue an audible alert to the user. One or more embodiments can include: computing a recommended break duration for the user; performing a text-to-speech process on a numerical value corresponding to the recommended break duration; generating audio data that includes speech of the numerical value corresponding to the recommended break duration; and sending the audio data to the CAE device for output thereon. In one or more embodiments, the determination of a recommended break duration can be based on one or more in-ear usage parameters, such as in-ear duration, inner ear temperature, inner ear humidity, and/or other in-ear usage parameters.

The user interface 501 can include an option to ventilate the ear canal during silent periods, which option is indicated at 516 as selected. A cancel option 522, when invoked, discards unsaved settings of the user interface of FIG. 5, and exits the user interface. A save option 524, when invoked, saves the settings of the user interface of FIG. 5 to memory, and exits the user interface. More, fewer, and/or different options may be present in one or more embodiments.

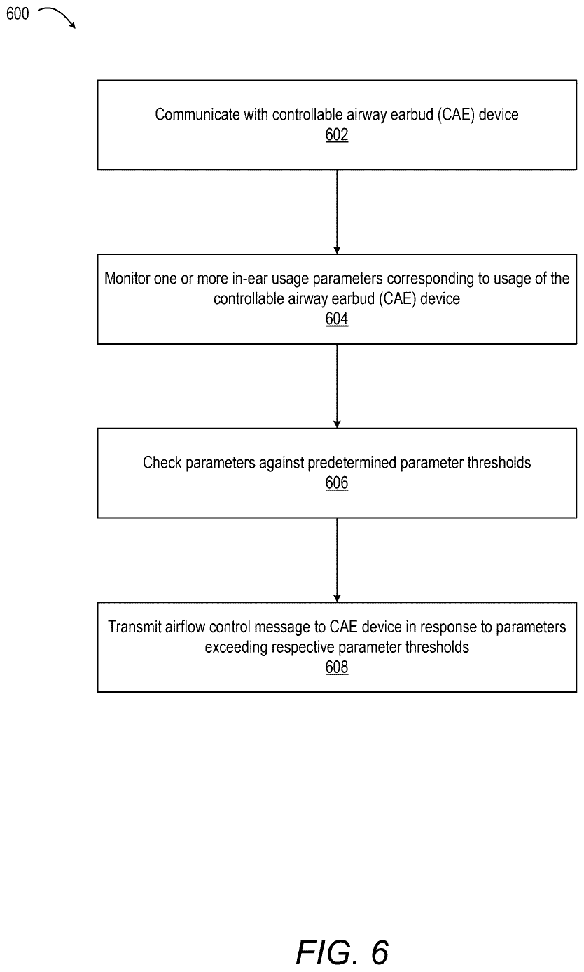

Referring now to the flowcharts presented by FIG. 6-FIG. 11, the descriptions of the methods in FIG. 6-FIG. 11 are provided with general reference to the specific components and features illustrated within the preceding FIG. 1-5. Specific components referenced in the methods of FIG. 6-FIG. 11 may be identical or similar to components of the same name used in describing preceding FIG. 1-5. In one or more embodiments, processor 112 (FIG. 1A) configures electronic device 100 (FIG. 1A) to provide the described functionality of the methods of FIG. 6-FIG. 11 by executing program code for one or more modules or applications provided within system memory 120 of electronic device 100, including ear health monitoring (EHM) module 125.

FIG. 6 depicts a flowchart of a computer-implemented method for managing airflow with a controllable airway earbud system, according to one or more embodiments. The method 600 starts at block 602, where an electronic device communicates with a controllable airway earbud (CAE) device. In one or more embodiments, the communication between the electronic device and the CAE device can be performed via Bluetooth, Bluetooth Low Energy, ultra-wideband (UWB), WiFi, and/or other suitable protocols and/or standards. The method 600 continues to block 604 where one or more in-ear usage parameters corresponding to usage of the CAE device are monitored. The in-ear usage parameters can include an in-ear duration, indicative of how long the CAE devices have been continuously worn. The in-ear usage parameters can include an in-ear temperature, indicative of an air temperature within the inner ear canal, and/or a surface temperature of a surface of the inner ear canal. The in-ear usage parameters can include an in-ear humidity level, indicative of moisture within the inner ear canal. The in-ear usage parameters can include an audio state of a user (wearer of the CAE device) as speaking or non-speaking. The in-ear usage parameters can include a noise cancellation mode, such as an active noise cancellation mode (ANC) setting. Other in-ear usage parameters may be monitored in one or more embodiments.

The method 600 continues to block 606 where the monitored in-ear usage parameters are checked against predetermined parameter thresholds. As an example, the safe maximum temperature of the inner ear canal typically aligns with normal body temperature ranges, as the ear canal is a sensitive area connected to the body's thermoregulatory system. A healthy temperature in the ear canal generally falls between 36.1 degrees Celsius and 37.9 degrees Celsius. Accordingly, in one or more embodiments, the predetermined parameter threshold for in-ear temperature can be established as 38 degrees Celsius. Other in-ear usage parameters can also have corresponding parameter thresholds. In some embodiments, the parameter thresholds may be user configurable through a user interface of the electronic device.

The method 600 continues to block 608 where an airflow control message is transmitted to the CAE device in response to in-ear usage parameters exceeding respective parameter thresholds. Referring again to the example of inner ear canal temperature, in response to detecting an inner ear canal temperature exceeding 38 degrees Celsius, an airflow control message can be sent to the CAE device to enable ventilation of the ear canal, such as depicted in FIG. 2B, in order to reduce the temperature in the inner ear canal.

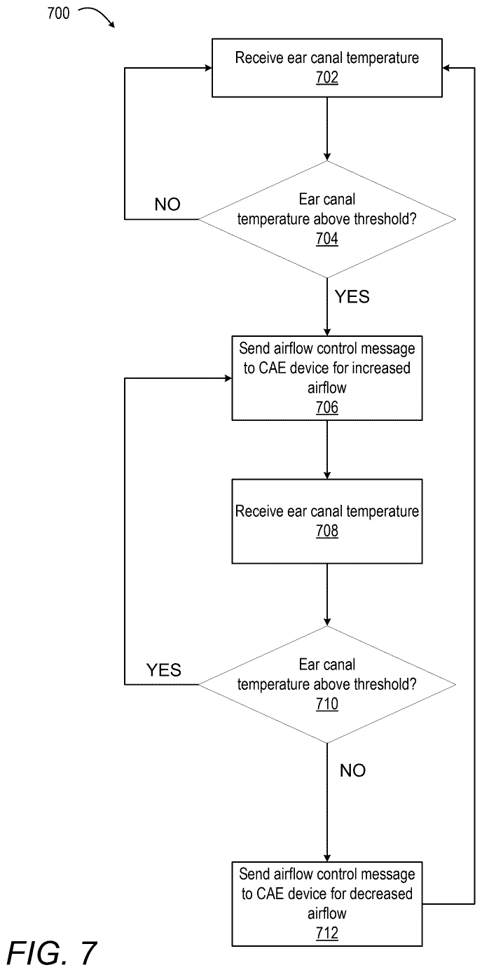

FIG. 7 depicts a flowchart of a computer-implemented method for managing airflow with a controllable airway earbud system based on ear canal temperature, according to one or more embodiments. The method 700 starts at block 702, where an inner ear canal temperature is received. In one or more embodiments, electronic device may periodically receive an ear canal temperature reading from a temperature sensor in the CAE device. In embodiments, an offset can be applied to the sensor reading to account for potential heat generated by the CAE itself. The acquired temperature data is temporarily stored in the CAE memory (284 of FIG. 2A). One or more embodiments can utilize Bluetooth Low Energy (BLE) to send temperature data to the paired electronic device. One or more embodiments may utilize a custom BLE service that provides characteristics for temperature data that includes a current temperature and a timestamp.

The method 700 continues to block 704, where a check is made to determine if the inner ear canal temperature is above a predetermined threshold (e.g., 38 degrees Celsius). If, at block 704, the inner ear canal temperature is not above the predetermined threshold, the method 700 returns to block 702 for continued monitoring. If, at block 704, the inner ear canal temperature is above the predetermined threshold, the method 700 continues to block 706 to send an airflow control message to the CAE device for reconfiguring the CAE device to provide increased airflow into the ear canal, such as depicted in FIG. 2B. The method 700 then continues to block 708, where an in-ear temperature is received. The method 700 then continues to block 710, where again a check is made to determine if the inner ear canal temperature is above a predetermined threshold (e.g., 38 degrees Celsius). If, at block 710, the inner ear canal temperature is above the predetermined threshold, the method 700 continues to block 706 where an airflow control message is sent to the CAE device for increased airflow. If, at block 710, the inner ear canal temperature is not above the predetermined threshold, the method 700 continues to block 712 to send an airflow control message to the CAE device for decreased airflow, such as depicted in FIG. 2A. In one or more embodiments, the smart fabric covering the earbud (e.g., 291 of FIG. 2C) is configured to have a smoother texture, thereby forming a more complete seal against the inner ear, reducing the effects of outside noise, and thus providing improved sound quality. One or more embodiments can include: receiving ear canal temperature data of an ear canal of the user from the CAE device; and in response to detecting the ear canal temperature exceeds a temperature threshold, triggering the transmitting of the airflow control message to the CAE device.

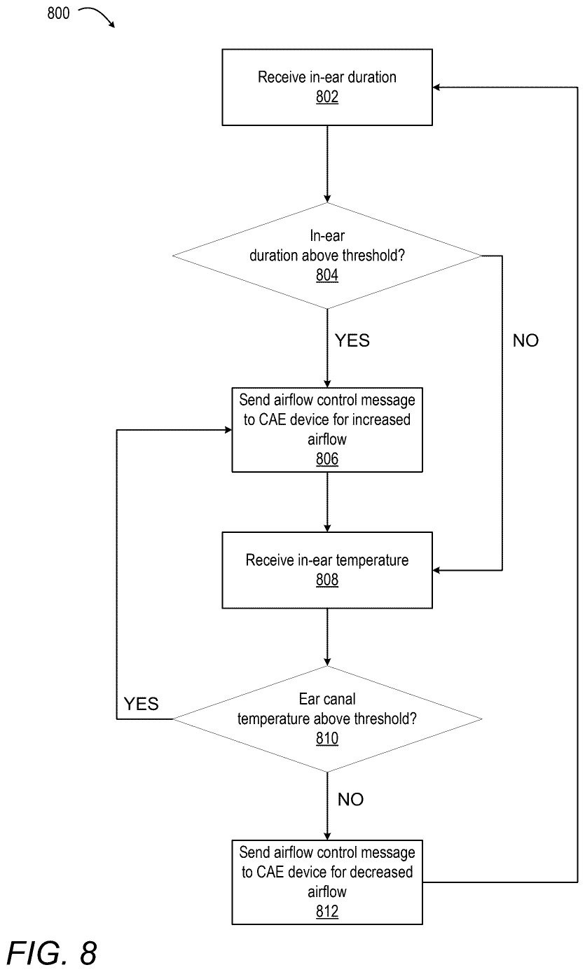

FIG. 8 depicts a flowchart of a computer-implemented method for managing airflow with a controllable airway earbud system based on in-ear duration, according to one or more embodiments. The method 800 starts at block 802, where an in-ear duration is received. In one or more embodiments, the electronic device may periodically receive an in-ear duration from the CAE device. In one or more embodiments, the CAE device can include an optical and/or infrared sensor to detect when the CAE device is inserted into an ear, as shown in FIG. 2A and/or FIG. 2B. One or more embodiments can utilize Bluetooth Low Energy (BLE) to send an in-ear status to the paired electronic device.

The method 800 continues to block 804, where a check is made to determine if in-ear duration is above a predetermined threshold. If, at block 804, the in-ear duration is not above the predetermined threshold, the method 800 returns to block 802 for continued monitoring. If, at block 804, the in-ear duration is above the predetermined threshold, the method 800 continues to block 806 to send an airflow control message to configure the CAE device for triggering increased airflow, such as depicted in FIG. 2B. The method 800 then continues to block 808, where an in-ear temperature is received. The method 800 then continues to block 810, where a check is made to determine if the in-ear temperature is above a predetermined threshold. If, at block 810, the in-ear duration is above the predetermined threshold, the method 800 continues to block 806 where an airflow control message is sent to the CAE device for increased airflow. Wearing earbuds for prolonged periods can be detrimental to ear health. Disclosed embodiments serve to mitigate this issue by increasing ventilation to the inner ear canal when the earbuds have been worn for an extended period. If, at block 810, the in-ear temperature is not above the predetermined threshold, the method 800 continues to block 812 to send an airflow control message to the CAE device to configure the CAE device for decreased airflow, such as depicted in FIG. 2A. One or more embodiments can include: determining an in-ear duration for the CAE device; and in response to the in-ear duration exceeding an in-ear duration: sending the airflow control message to the CAE device to trigger the adjustment in the seal of the CAE device.

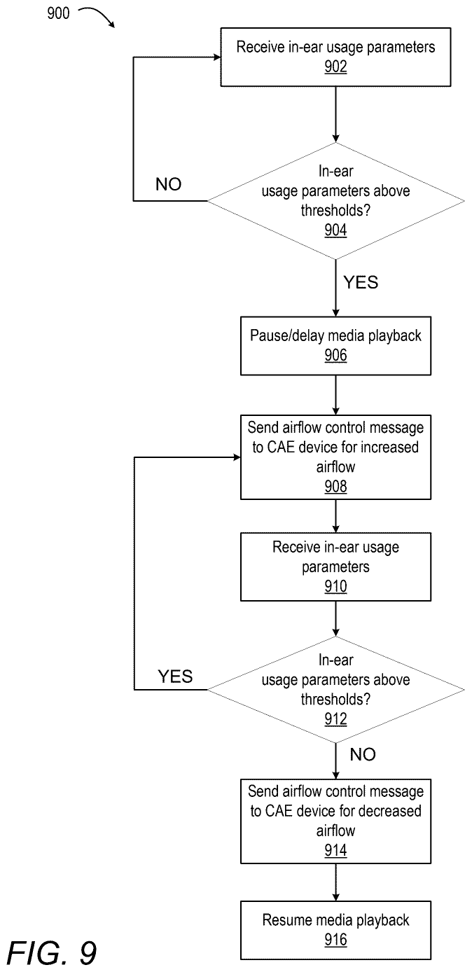

FIG. 9 depicts a flowchart of a computer-implemented method for managing airflow with a controllable airway earbud system based on pausing media playback, according to one or more embodiments. The method 900 starts at block 902, where in-ear usage parameters are received from the CAE device. The method continues to block 904, where a check is made to determine if the in-ear usage parameters are above corresponding predetermined thresholds. If, at block 904, the in-ear usage parameters are not above the corresponding predetermined thresholds, the method 900 returns to block 902 for continued monitoring. If, at block 904, the in-ear usage parameters are above the corresponding predetermined thresholds, the method 900 continues to block 906 to pause/delay media playback, such as depicted in FIG. 3B. The method 900 continues to block 908, to send an airflow control message to the CAE device for increased airflow, such as depicted in FIG. 2B. The method 900 continues to block 910 to continue to receive in-ear usage parameters. The method 900 continues to block 912 where a check is again made to determine if the in-ear usage parameters are above corresponding predetermined thresholds. If, at block 912, the in-ear usage parameters are above the corresponding predetermined thresholds, the media remains paused, and the ventilation is increased, and the method 900 returns to block 908 for sending an airflow control message to the CAE device for increased airflow. If, at block 912, the in-ear usage parameters are not above the corresponding predetermined thresholds, the method 900 continues to block 914, to send an airflow control message to the CAE device for decreased airflow, such as depicted in FIG. 2A. The method 900 then continues to block 916 to resume media playback, such as depicted at 366 of FIG. 3B. One or more embodiments can include: pausing playback of media that is being sent to the CAE device prior to sending the airflow control message to adjust the seal of the CAE device, where the airflow control message to the CAE device causes the CAE device to increase airflow to the corresponding ear canal of the user. In general, disclosed embodiments function similarly for both the left ear and the right ear. After a period of time with the CAE device configured for increased ventilation, the CAE seal can be reconfigured to reduce ventilation, which also improves sound quality by reducing the amount of outside noise that the user can hear. One or more embodiments can further include: sending a second airflow control message that causes the CAE device to decrease airflow to the corresponding ear canal after a time duration; and resuming playback of the media that is being sent to the CAE device.

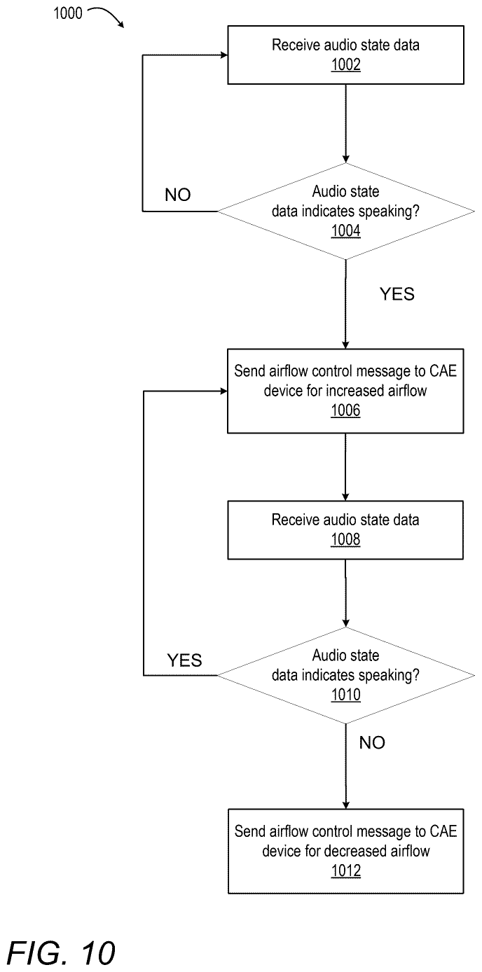

FIG. 10 depicts a flowchart of a computer-implemented method for managing airflow with a controllable airway earbud system based on audio state data, according to one or more embodiments. The method 1000 starts at block 1002, where audio state data is received from the CAE device. The audio state data can include data indicative of the voice of the user being detected by a microphone of the CAE device. Hence, the audio state data can be indicative of the user having an audio state of speaking (when speech data is detected by the microphone) or non-speaking (when no speech data is detected by the microphone). The method 1000 continues to block 1004, where a check is made to determine if the audio state data indicates a speaking state. If, at block 1004, the audio state data does not indicate a speaking state, the method 1000 returns to block 1002 for continued monitoring. If, at block 1004, the audio state data indicates a speaking state, the method 1000 continues to block 1006, where the method includes sending an airflow control message to the CAE device for increased airflow, such as depicted in FIG. 2B. The method 1000 continues to block 1008, to again receive audio state data from the CAE device, similar to as described for block 1002. The method 1000 continues to block 1010, where again a check is made to determine if the audio state data indicates a speaking state. If, at block 1010, the audio state data indicates a speaking state, the method 1000 returns to block 1006 for sending an airflow control message to the CAE device for increased airflow. If, at block 1010, the audio state data does not indicate a speaking state, the method 1000 continues to block 1012, to send an airflow control message to the CAE device for decreased airflow, such as depicted in FIG. 2A. One or more embodiments can include: determining, based on audio state data, an audio state of the user as speaking; and in response to determining the audio state of the user as speaking, sending an airflow control message to the CAE device that causes the CAE device to increase airflow to the corresponding ear canal of the user while the user is speaking. One or more embodiments can further include: determining, based on the audio state data, an audio state of the user as non-speaking; and in response to determining the audio state of the user as non-speaking, sending an airflow control message to the CAE device that causes the CAE device to decrease airflow to the corresponding ear canal of the user.

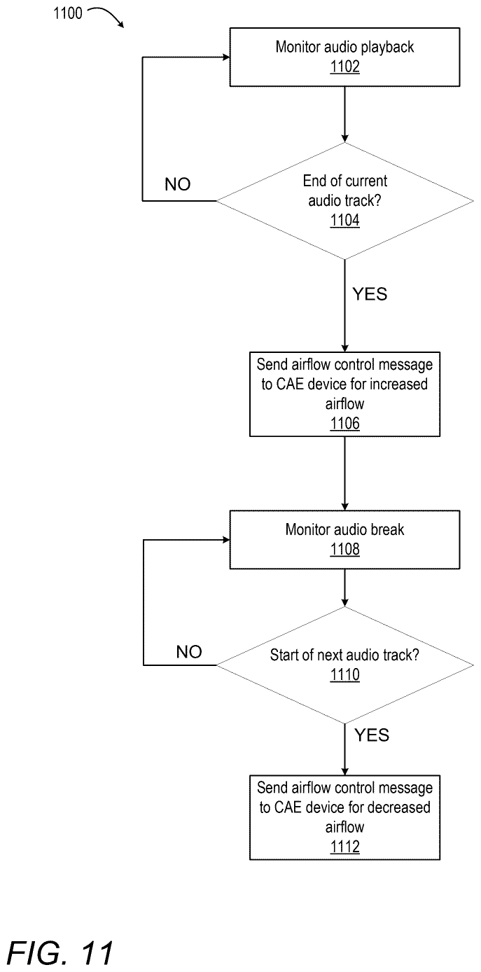

FIG. 11 depicts a flowchart of a computer-implemented method for managing airflow with a controllable airway earbud system based on silence breaks between audio tracks, according to one or more embodiments. The method 1100 starts at block 1102, where audio playback from the electronic device is monitored. The method 1100 continues to block 1104, where a check is made to determine if the audio track has ended. If, at block 1104, the audio track has not ended, the method 1100 returns to block 1102 for continued monitoring. If, at block 1104, the audio track has ended (such as may be indicated at 304 in FIG. 3A), the method 1100 continues to block 1106, at which method 1100 includes sending an airflow control message to the CAE device for increased airflow, such as depicted in FIG. 2B. The method 1100 continues to block 1108, to monitor the audio break (e.g., such as indicated at 304 of FIG. 3A). The method 1100 continues to block 1110, where a check is made to determine if the next audio track has started. If, at block 1110, the next audio track has not started, the method 1100 returns to block 1108 for continued monitoring. If, at block 1010, the next audio track has started, the method 1100 continues to block 1112, to send an airflow control message to the CAE device for decreased airflow, such as depicted in FIG. 2A

The flowcharts, sequences, and configurations presented herein are provided solely for illustrative purposes and are exemplary in nature. These embodiments are not intended to be limiting and may include variations with more, fewer, and/or alternative options, sequences, or features as would be apparent to those skilled in the art.

As can now be appreciated, disclosed embodiments provide techniques for managing airflow in an ear canal with a controllable airway earbud system. Disclosed embodiments interface with a CAE device to cause the CAE device to periodically ventilate the inner ear canal, offering significant advantages for promoting ear health. By actively managing factors such as temperature and humidity, disclosed embodiments can mitigate the risks associated with prolonged earbud use. Prolonged use of earbuds can trap moisture in the ear canal, creating an environment conducive to bacterial and fungal growth, which can lead to infections such as otitis externa (swimmer's ear). The features provided by disclosed embodiments periodically introduce airflow to reduce moisture levels in the ear canal, disrupting conditions that encourage microbial growth. Moreover, the ear canal can become warm during extended earbud use, leading to discomfort or irritation. The controllable airway enabled by disclosed embodiments can dissipate heat and maintain a more natural temperature balance, improving comfort during long listening sessions.

Additionally, warm, humid environments in the ear canal can lead to sensations of fullness, irritation, or itching, reducing the user's ability to wear earbuds comfortably for extended periods. Ventilation provided by disclosed embodiments enables the ear canal to remain closer to its natural state in terms of temperature and humidity, preventing discomfort and allowing users to wear earbuds longer without adverse effects. Thus, disclosed embodiments provide a unique combination of comfort, performance, and health benefits. By enabling periodic ventilation based on various conditions to regulate humidity and temperature, disclosed embodiments can serve to reduce infections and discomfort associated with prolonged use of earbuds.

In the above-described methods, one or more of the method processes may be embodied in a computer readable device containing computer readable code such that operations are performed when the computer readable code is executed on a computing device. In some implementations, certain operations of the methods may be combined, performed simultaneously, in a different order, or omitted, without deviating from the scope of the disclosure. Further, additional operations may be performed, including operations described in other methods. Thus, while the method operations are described and illustrated in a particular sequence, use of a specific sequence or operations is not meant to imply any limitations on the disclosure. Changes may be made with regards to the sequence of operations without departing from the spirit or scope of the present disclosure. Use of a particular sequence is therefore, not to be taken in a limiting sense, and the scope of the present disclosure is defined primarily by the appended claims.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. Computer program code for carrying out operations for aspects of the present disclosure may be written in any combination of one or more programming languages, including an object-oriented programming language, without limitation. These computer program instructions may be provided to a processor of a general-purpose computer, special-purpose computer, or other programmable data processing apparatus to produce a machine that performs the method for implementing the functions/acts specified in the flowchart and/or block diagram block or blocks. The methods are implemented when the instructions are executed via the processor of the computer or other programmable data processing apparatus.

As will be further appreciated, the processes in embodiments of the present disclosure may be implemented using any combination of software, firmware, or hardware. Accordingly, aspects of the present disclosure may take the form of an entirely hardware embodiment or an embodiment combining software (including firmware, resident software, micro-code, etc.) and hardware aspects that may all generally be referred to herein as a “circuit,” “module,” or “system.” Furthermore, aspects of the present disclosure may take the form of a computer program product embodied in one or more computer readable storage device(s) having computer readable program code embodied thereon. Any combination of one or more computer readable storage device(s) may be utilized. The computer readable storage device may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage device can include the following: a portable computer diskette, a hard disk, a random-access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage device may be any tangible medium that can contain or store a program for use by or in connection with an instruction execution system, apparatus, or device.

Where utilized herein, the terms “tangible” and “non-transitory” are intended to describe a computer-readable storage medium (or “memory”) excluding propagating electromagnetic signals, but are not intended to otherwise limit the type of physical computer-readable storage device that is encompassed by the phrase “computer-readable medium” or memory. For instance, the terms “non-transitory computer readable medium” or “tangible memory” are intended to encompass types of storage devices that do not necessarily store information permanently, including, for example, RAM. Program instructions and data stored on a tangible computer-accessible storage medium in non-transitory form may afterwards be transmitted by transmission media or signals such as electrical, electromagnetic, or digital signals, which may be conveyed via a communication medium such as a network and/or a wireless link.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

As used herein, the term “or” is inclusive unless otherwise explicitly noted. Thus, the phrase “at least one of A, B, or C” is satisfied by any element from the set {A, B, C} or any combination thereof, including multiples of any element.

While the disclosure has been described with reference to example embodiments, it will be understood by those skilled in the art that various changes may be made and equivalents may be substituted for elements thereof without departing from the scope of the disclosure. In addition, many modifications may be made to adapt a particular system, device, or component thereof to the teachings of the disclosure without departing from the scope thereof. Therefore, it is intended that the disclosure not be limited to the particular embodiments disclosed for carrying out this disclosure, but that the disclosure will include all embodiments falling within the scope of the appended claims.

What is claimed is:

1. An electronic device comprising:
a communications subsystem enabling the electronic device to communicatively connect to a controllable airway earbud (CAE) device;
a media subsystem enabling the electronic device to send transmitted audio data to and receive audio data from the CAE device;
a memory having stored thereon an ear health monitoring (EHM) module; and
at least one processor coupled to the communications subsystem, the media subsystem, and the memory and which processes program code of the EHM module, the at least one processor configured to cause the electronic device to:
monitor one or more in-ear usage parameters corresponding to usage of the CAE device by a user; and
in response to a monitored in-ear usage parameter exceeding a respective parameter threshold, transmit an airflow control message to the CAE device to trigger an adjustment in a seal of the CAE device and enable airflow into a corresponding ear canal of the user.

2. The electronic device of claim 1, wherein the one or more in-ear usage parameters comprise an ear canal temperature, and the at least one processor is further configured to cause the electronic device to:
receive ear canal temperature data of an ear canal of the user from the CAE device; and
in response to detecting the ear canal temperature exceeds a temperature threshold, trigger transmitting of the airflow control message to the CAE device.

3. The electronic device of claim 1, wherein the one or more in-ear usage parameters comprise an in-ear duration and the at least one processor is further configured to cause the electronic device to:
determine the in-ear duration for the CAE device; and
in response to the in-ear duration exceeding an in-ear duration threshold:
send the airflow control message to the CAE device to trigger the adjustment in the seal of the CAE device.

4. The electronic device of claim 1, wherein the at least one processor is further configured to cause the electronic device to:
pause playback of media that is being sent to the CAE device prior to sending the airflow control message to adjust the seal of the CAE device, wherein the airflow control message to the CAE device causes the CAE device to increase airflow to the corresponding ear canal of the user.

5. The electronic device of claim 4, wherein the at least one processor is further configured to cause the electronic device to:
send a second airflow control message that causes the CAE device to decrease airflow to the corresponding ear canal after a duration; and
resume playback of the media that is being sent to the CAE device.

6. The electronic device of claim 1, wherein the at least one processor is further configured to cause the electronic device to:
determine, based on audio state data, an audio state of the user as speaking; and
in response to determining the audio state of the user as speaking, send an airflow control message to the CAE device that causes the CAE device to increase airflow to the corresponding ear canal of the user while the user is speaking.

7. The electronic device of claim 6, wherein the at least one processor is further configured to cause the electronic device to:
determine, based on the audio state data, an audio state of the user as non-speaking; and
in response to determining the audio state of the user as non-speaking, send an airflow control message to the CAE device that causes the CAE device to decrease airflow to the corresponding ear canal of the user.

8. The electronic device of claim 1, wherein the at least one processor is further configured to cause the electronic device to:
in response to detecting an ear canal temperature exceeding a second predetermined threshold, send an auditory warning message to the CAE device that causes the CAE device to issue an audible alert to the user.

9. The electronic device of claim 8, wherein the at least one processor is further configured to cause the electronic device to:
compute a recommended break duration for the user;
perform a text-to-speech process on a numerical value corresponding to the recommended break duration;
generate audio data that includes speech of the numerical value corresponding to the recommended break duration; and
send the audio data to the CAE device.

10. The electronic device of claim 1, wherein the at least one processor is further configured to cause the electronic device to:
monitor a current audio playback state of the CAE device; and
in response to detecting a pause or end in presentation of audio output by the CAE device, generate and send another airflow control message to the CAE device that causes the CAE device to increase airflow to an ear canal of the user during the pause or end in presentation of the audio output.

11. The electronic device of claim 1, wherein the at least one processor is further configured to cause the electronic device to:
detect a first characteristic of the CAE device that enables reconfiguration of at least one external surface of the CAE device to allow air flow past the CAE device into the corresponding ear canal; and
select an airflow control message to send to the CAE device to adjust a seal of the CAE device, in response to detecting the first characteristic.

12. A method comprising:
monitoring, on an electronic device that includes a communications subsystem enabling the electronic device to communicatively connect to a controllable airway earbud (CAE) device, one or more in-ear usage parameters corresponding to usage of the CAE device by a user; and
in response to a monitored in-ear usage parameter exceeding a respective parameter threshold, transmitting an airflow control message to the CAE device to trigger an adjustment in a seal of the CAE device and enable airflow into a corresponding ear canal of the user.

13. The method of claim 12, further comprising:
receiving ear canal temperature data of an ear canal of the user from the CAE device; and
in response to detecting the ear canal temperature exceeds a temperature threshold, triggering the transmitting of the airflow control message to the CAE device.

14. The method of claim 12, further comprising:
determining an in-ear duration for the CAE device; and
in response to the in-ear duration exceeding an in-ear duration threshold:
sending the airflow control message to the CAE device to trigger the adjustment in the seal of the CAE device.

15. The method of claim 12, further comprising pausing playback of media that is being sent to the CAE device prior to sending the airflow control message to adjust the seal of the CAE device, wherein the airflow control message to the CAE device causes the CAE device to increase airflow to the corresponding ear canal of the user.

16. The method of claim 15, further comprising:
sending a second airflow control message that causes the CAE device to decrease airflow to the corresponding ear canal after a duration; and
resuming playback of the media that is being sent to the CAE device.

17. The method of claim 12, further comprising:
determining, based on audio state data, an audio state of the user as speaking; and
in response to determining the audio state of the user as speaking, sending an airflow control message to the CAE device that causes the CAE device to increase airflow to the corresponding ear canal of the user while the user is speaking.

18. The method of claim 17, further comprising:
determining, based on the audio state data, an audio state of the user as non-speaking; and
in response to determining the audio state of the user as non-speaking, sending an airflow control message to the CAE device that causes the CAE device to decrease airflow to the corresponding ear canal of the user.

19. The method of claim 12, further comprising:
in response to detecting an ear canal temperature exceeding a second predetermined threshold, sending an auditory warning message to the CAE device that causes the CAE device to issue an audible alert to the user.

20. A computer program product comprising a non-transitory computer readable medium having program instructions that when executed by a processor of an electronic device comprising a communications subsystem enabling the electronic device to communicatively connect to a controllable airway earbud (CAE) device, configure the electronic device to perform functions comprising:
monitoring one or more in-ear usage parameters corresponding to usage of the CAE device by a user; and
in response to a monitored in-ear usage parameter exceeding a respective parameter threshold, transmitting an airflow control message to the CAE device to trigger an adjustment in a seal of the CAE device and enable airflow into a corresponding ear canal of the user.
